(19) United States
(12) Patent Application Publication
(10) Pub. No.: US 2026/0140076 A1
(43) Pub. Date: May 21, 2026

(54) MEASUREMENT DEVICE AND METHOD FOR THERMAL CHARACTERISTICS OF FROZEN SOIL BASED ON THERMAL PULSE METHOD

(71) Applicant: Ludong University, Yantai (CN)

(72) Inventors: Ying Zhao, Yantai (CN); Xiaolong Wu, Yantai (CN); Yi Wang, Yantai (CN); Zhongen Niu, Yantai (CN); Haixia Wang, Yantai (CN)

(21) Appl. No.: 19/011,961

(22) Filed: Jan. 7, 2025

(30) Foreign Application Priority Data
Nov. 19, 2024 (CN) 2024116555668

(51) Int. Cl. G01N 25/18; G01N 33/24

(52) U.S. Cl. CPC G01N 25/18; G01N 33/24

(57) ABSTRACT

A measurement device and method for thermal characteristics of frozen soil based on a thermal pulse method, comprising heating the cylindrical heating probe with the heater at a constant power; the first temperature probe detects the temperature of the cylindrical heating probe and uploads it to the cloud via the transmission unit, while data recorder displays the temperature data of the first temperature probe and the second temperature probe in real-time; the temperature of the cylindrical heating probe is maintained at a constant level; by heating the frozen soil sample with the cylindrical heating probe, the temperature breakthrough curve of the frozen soil sample is obtained, then the volumetric diffusivity and volumetric thermal conductivity of the frozen soil sample is obtained. This invention can effectively keep the maximum spatial temperature below the freezing point, thereby enabling the estimation of the overall thermal characteristics of quartz sand and ice content.

### 1. TECHNICAL FIELD

The invention relates to the technical field of frozen soil freezing and thawing, in particular to a measurement device and method for thermal characteristics of frozen soil based on a thermal pulse method.

### 2. BACKGROUND ART

The freeze-thaw process is crucial for quantifying the soil surface energy balance and its interaction with hydrological processes. In the past few decades, significant progress has been made in developing methods to measure soil ice content and improving measurement accuracy. Thermal resistance temperature probes, differential scanning calorimetry, TDR (time-domain reflectometry), thermal pulse methods, and nuclear magnetic resonance (NMR) have all enabled more precise measurements of soil ice content in frozen soils. However, the aforementioned methods still face some challenges, such as the melting and refreezing of ice, which can transfer a significant portion of energy to phase change rather than to heat conduction; although some researchers have adopted adjustments to address these problems, such as assuming minimal ice melting, integrating latent heat flux into calculations, or using numerical models, these adjustments may lead to biased or inaccurate estimates of thermal characteristics, especially in partially frozen soils.

Due to the lack of methods for measuring soil thermal characteristics under freezing or melting conditions, characterizing soil freezing and thawing processes remains hindered. When using existing thermal pulse methods, heat is released in a relatively short time and/or with high heat flux to ensure the accuracy of detection signals. The temperature increase on the probe surface may exceed the freezing point, leading to the melting of frozen soil, making it impossible to accurately obtain soil thermal characteristics within the temperature range of −5° C. to 0° C.

### 3. SUMMARY OF THE INVENTION

In order to solve the problem that the temperature increase on the probe surface of the heat pulse method in the prior art may exceed the freezing point, leading to the melting of frozen soil, making it impossible to accurately obtain soil thermal characteristics within the temperature range of −5° C. to 0° C., the invention provides a measurement device and method for thermal characteristics of frozen soil based on a thermal pulse method.

In order to achieve the above objects, the invention adopts the following technical scheme:
a measurement device for thermal characteristics of frozen soil based on a thermal pulse method comprises a testing assembly, a variable voltage control device, and a data processing device;
the testing assembly comprises a cylindrical heating probe, a first temperature probe, and a second temperature probe; the cylindrical heating probe, the first temperature probe, and the second temperature probe are all placed in the frozen soil sample to be measured; the variable voltage control device is connected to the cylindrical heating probe and the first temperature probe, providing energy to the cylindrical heating probe, and the first temperature probe records the surface temperature of the heating probe; the data processing device is connected to the second temperature probe; the second temperature probe measures the temperature changes of the frozen soil.

Further, the first temperature probe, the second temperature probe, and the cylindrical heating probe are arranged parallel to each other; the first temperature probe, the second temperature probe, and the cylindrical heating probe are arranged in the frozen soil sample to be measured in either a horizontal or vertical manner.

Further, the data processing device comprises a data recorder, a processing unit, a transmission unit, and a cloud; the processing unit is connected to the cloud; the cloud is connected to the transmission unit, the data recorder and the transmission unit are both electrically connected to the first temperature probe and the second temperature probe; the transmission unit sends the collected data from the first temperature probe and the second temperature probe to the cloud for storage; the processing unit retrieves and analyzes the stored data, and then stores the processed data again in the cloud; the data recorder displays the temperature data of the first temperature probe and the second temperature probe.

Further, the variable voltage control device comprises a heater and a temperature controller; the heater is connected to the cylindrical heating probe through the temperature controller.

A measurement method for thermal characteristics of frozen soil based on the thermal pulse method uses the measurement device for thermal characteristics of frozen soil based on the thermal pulse method, wherein the measurement method comprises:
based on the constant temperature heating of the cylindrical heating probe by the heater, the first temperature probe detects the temperature of the cylindrical heating probe, and the second temperature probe detects the temperature of the frozen soil, the data is then uploaded to the cloud through the transmission unit, while the data recorder displays the temperature data of the cylindrical heating probe and the frozen soil in real-time;
when the cylindrical heating probe reaches the target temperature, the temperature controller maintains the temperature of the cylindrical heating probe at a constant value;
under the heating of the cylindrical heating probe, a temperature breakthrough curve of the frozen soil sample and energy received by the frozen soil sample are obtained, thereby obtaining thermal diffusivity of the frozen soil sample;
based on heating time of the cylindrical heating probe and the heat output during that heating time, total heat output of the cylindrical heating probe is calculated;
based on the energy received by the frozen soil sample, total delivered energy of the cylindrical heating probe, and volumetric diffusivity of the frozen soil sample, volumetric heat capacity is obtained;
based on the thermal diffusivity and volumetric heat capacity, thermal conductivity is obtained.

Further, when the cylindrical heating probe controls the energy output power of the heater through the temperature controller to keep the temperature of the cylindrical heating probe constant, specifically: when the temperature reading of the cylindrical heating probe drops to the preset lower temperature limit, the heater power supply is turned on, and heating is performed by using short pulses with low energy output power; after the heating period ends, the heater power supply is turned off to keep the temperature of the cylindrical heating probe between the preset upper and lower temperature limits; this step is continuously repeated until the frozen soil sample measurement is completed.

Further, the temperature breakthrough curve of the frozen soil sample is obtained as follows:
one-dimensional transient heat conduction process in the cylindrical coordinate system is described as follows:

$$\frac{{\partial }^{2} T}{\partial {r}^{2}} + \frac{1}{r} \frac{\partial T}{\partial r} = \frac{1}{D} \frac{\partial T}{\partial t} ( r > {r}_{e} , t > 0 )$$

wherein T is temperature measured in the soil by the second temperature probe; r is a radial distance from the second temperature probe to the cylindrical heating probe; t is time for measuring the temperature breakthrough curve of the frozen soil; effective radius rₑ is a radial distance between the second temperature probe and the first temperature probe; minimum value of rₑ is equal to the sum of the radii of the first temperature probe and the second temperature probe; and D is volumetric thermal diffusivity;
when heating the frozen soil by using a constant-temperature heat source, target temperature T₁ of the frozen soil is higher than initial temperature T₀ but lower than the freezing point;
while satisfying both initial condition (T(r,0)=T₀(T₀<0)) and boundary condition (T(rₑ,t)=T₁(T₀<T₁<0)), the solution in Laplace space is as follows:

$$\overline{T} ( r , s ) = \frac{( {T}_{1} - {T}_{0} ) {K}_{0} ( q r )}{{sK}_{0} ( {qr}_{e} )}$$

wherein K₀ is zero-ordered modified Bessel function of the second kind;

$${q}^{2} = \frac{s}{D}$$

wherein s is time t in the Laplace space after is T(r,t) Laplace transformed;
by performing the numerical inverse Laplace transform, the spatial distribution T(r,t) of temperature in frozen soil at any time can be obtained;
Root Mean Square Error (RMSE) is used to evaluate the fitting of the temperature breakthrough curve, specifically:

$$RMSE = \sqrt{\frac{{\sum }_{i = 1}^{N} {( {T}_{i} - {\hat{T}}_{ι} )}^{2}}{N}}$$

wherein subscript i is the sequence of measured temperature responses; N is the total number of measured temperatures; T and \hat{T} respectively represent measured temperatures and predicted temperatures.

Further, the energy received by the frozen soil sample, specifically:

$${Q}_{1} ( t ) = {\sum }_{i = 1}^{M} ( {T}_{i} - {T}_{0} ) {ρ}_{b} {c}_{b} π ( {r}_{i + 1}^{2} - {r}_{i}^{2} )$$

wherein subscript i represents the i-th discrete spatial interval after the measurement space is discretized into M spatial intervals in the radial direction, with the spatial range covering the radial distance from r=rₑ to T=T₀;
based on the heating time of the cylindrical heating probe and the heat output during that heating time, the total heat output of the cylindrical heating probe is calculated, specifically:
total output energy Q₂(t) of the cylindrical heating probe is measured by the temperature controller; heating cycle consists of multiple pulsed heating intervals with a given power Pⱼ and heating period tpⱼ, so the total delivered energy is the sum of the energy delivered in each cycle, as shown below:

$${Q}_{2} ( t ) = \frac{1}{L} {\sum }_{j = 1}^{N} {P}_{j} t {p}_{j}$$

wherein L represents length of the cylindrical heating probe.

Further, based on the energy received by the frozen soil sample, the total delivered energy of the cylindrical heating probe, and the volumetric diffusivity of the frozen soil sample, the volumetric heat capacity is obtained, specifically:
from the volumetric heat capacity C_{b}=ρ_{b}c_{b}, the following formula can be obtained:

$${C}_{b} = \frac{{\sum }_{j = 1}^{N} {P}_{j} t {p}_{j}}{L {\sum }_{i = 1}^{M} ( {T}_{i} - {T}_{0} ) π ( {r}_{i + 1}^{2} - {r}_{i}^{2} )}$$

wherein ρ_{b} represents density of medium; c_{b} represents specific heat capacity of the medium;
based on the thermal diffusivity and the volumetric heat capacity, the thermal conductivity is obtained, specifically:
volumetric thermal conductivity of soil:

$${λ}_{b} = D {C}_{b} .$$

Further, when other substances are doped into the frozen soil, and in the case of quartz sand samples being doped, total heat capacity of the quartz sand samples is as follows:

$$C = {n}_{i} {c}_{i} + {n}_{g} {c}_{g} + {n}_{s} {c}_{s}$$

wherein n represents volume fraction of components in frozen soil, cᵢ represents specific heat capacity of ice; c_{g} represents specific heat capacity of air; and cₛ represents specific heat capacity of solid;
in saturated frozen soil, gaseous term is omitted; based on the total heat capacity C, as well as the specific heat capacities of the solid and ice, the respective volume fractions can be obtained.

Compared with the prior art, the invention has the following advantages:
the invention involves heating the cylindrical heating probe with the heater at a constant power; the first temperature probe detects the temperature of the cylindrical heating probe and uploads it to the cloud via the transmission unit, while data recorder displays the temperature data of the first temperature probe and the second temperature probe in real-time; the temperature of the cylindrical heating probe is maintained at a constant level; by heating the frozen soil sample with the cylindrical heating probe, the temperature breakthrough curve of the frozen soil sample is obtained, then the volumetric diffusivity and volumetric thermal conductivity of the frozen soil sample is obtained. This invention can effectively keep the maximum spatial temperature below the freezing point, thereby enabling the estimation of the overall thermal characteristics of quartz sand and ice content, and improving the accuracy of estimating the overall thermal characteristics of frozen soil samples in cold regions.

### 4. BRIEF DESCRIPTION OF ACCOMPANY DRAWINGS

In order to explain the technical schemes in the embodiments of the invention more clearly, the accompanying drawings needed to be used in the embodiments will be briefly introduced below. It should be understood that the following accompanying drawings only illustrate certain embodiments of the invention. Therefore, they should not be considered to limit the scope. For those of ordinary skill in the art, other accompanying drawings can be obtained based on these accompanying drawings without exerting creative efforts.

FIG. 1 is a schematic diagram of the topological structure of the measurement device for thermal characteristics of frozen soil based on a thermal pulse method of the invention.

FIG. 2 is a flow chart of the measurement method for thermal characteristics of frozen soil based on a thermal pulse method of the invention.

FIG. 3 is a schematic diagram of the constant temperature control process by using a multi-pulse heating strategy.

FIG. 4 is a schematic diagram of the spatial temperature distribution along the radial direction at different times t₁, t₂ and t₃ estimated by ILS model.

### 5. SPECIFIC EMBODIMENT OF THE INVENTION

In order to make the objects, technical schemes and advantages of the embodiments of the invention clearer, the technical schemes in the embodiments of the invention will be clearly and completely described below in combination with the accompanying drawings in the embodiments of the invention, obviously, the described embodiments are some, but not all embodiments of the invention. The components of the embodiments of the invention generally described and illustrated in the drawings herein may be arranged and designed in a variety of different configurations.

Therefore, the following detailed description of the embodiments of the invention provided in the accompanying drawings is not intended to limit the scope of the claimed invention, but merely represents selected embodiments of the invention. All other embodiments obtained by ordinary technicians in this field without creative work based on the embodiments of the invention are within the protection scope of the invention.

It should be noted that similar reference numerals and letters denote similar items in the following accompanying drawings. Therefore, once an item is defined in one accompanying drawing, further definition and explanation thereof is not required in subsequent accompanying drawings.

In the description of the invention, it should be understood that the orientation or positional relationship indicated by the terms “upper”, “lower”, “horizontal”, “inner” and so on are based on the orientation or positional relationship shown in the accompanying drawings, or the orientation or positional relationship that product of the invention is usually placed in when it is used, only for the convenience of describing the invention and simplifying the description, rather than indicating or implying that the indicated device or element must have a specific orientation, as well as a specific orientation structure and operation, therefore, it should not be construed as a limitation of the invention. In addition, the terms of “first”, “second”, “third” and so on are only used to differentiate the description, and should not be construed as indicating or implying relative importance.

In addition, if the terms “horizontal” appear, it does not mean that the component is required to be absolutely horizontal, but can be slightly inclined. For example, “horizontal” simply means that its direction is more horizontal than “vertical”, it does not mean that the structure must be completely horizontal, but can be slightly inclined.

In the description of the invention, it should also be noted that the terms of “arrange”, “install”, “link”, “connect”, etc., should be generally understood unless there are specific restrictions or stipulations, for example, the “connect” may refer to fixed connection, detachable connection or integral connection; the “connect” may also refer to mechanical connection or electrical connection; the means of “connect” may be directly connected or indirectly connected through an intermediate medium, and may be internal communication between the two elements. For those skilled in the art, the specific meaning of the above terms in the invention can be understood according to the specific situation.

The invention is further described in detail below in combination with the accompanying drawings.

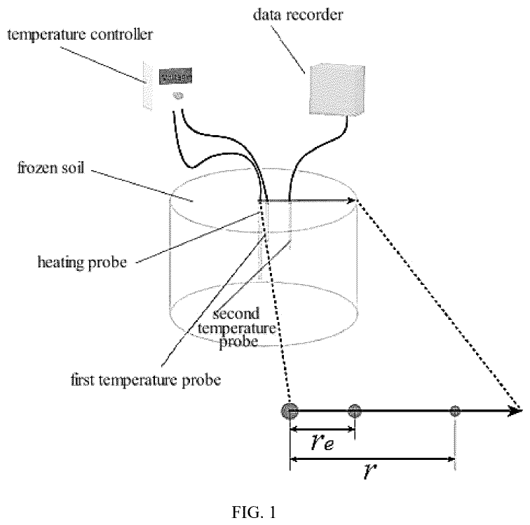

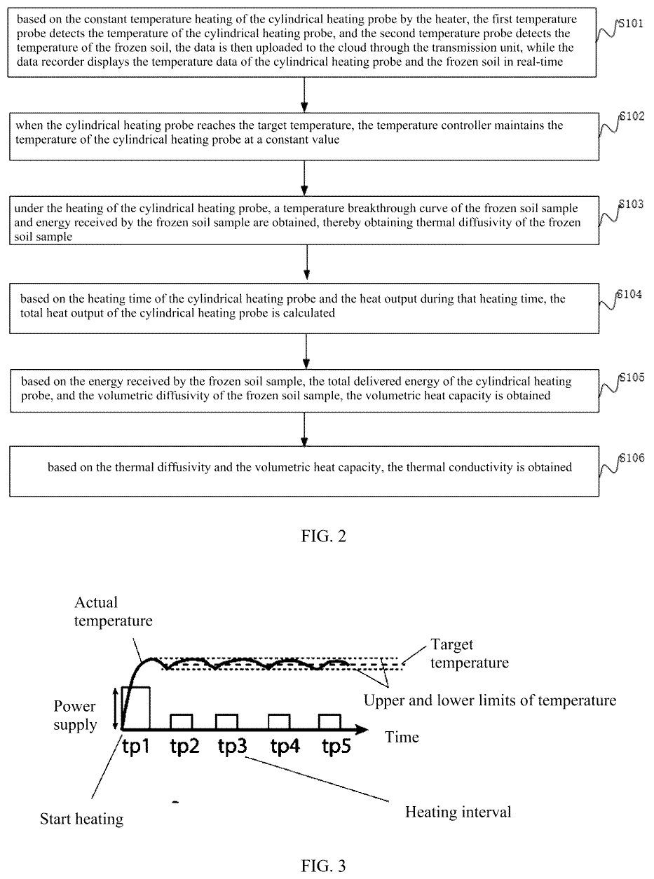

Referring to FIG. 1, a measurement device for thermal characteristics of frozen soil based on a thermal pulse method comprises a testing assembly, a variable voltage control device, and a data processing device;
the testing assembly comprises a cylindrical heating probe, a first temperature probe, and a second temperature probe; the cylindrical heating probe, the first temperature probe, and the second temperature probe are all placed in the frozen soil sample to be measured; the variable voltage control device is connected to the cylindrical heating probe and the first temperature probe, providing energy to the cylindrical heating probe, and the first temperature probe records the surface temperature of the heating probe; the data processing device is connected to the second temperature probe; the second temperature probe measures the temperature changes of the frozen soil.

The first temperature probe, the second temperature probe, and the cylindrical heating probe are arranged parallel to each other; the first temperature probe, the second temperature probe, and the cylindrical heating probe are arranged in the frozen soil sample to be measured in either a horizontal or vertical manner.

The data processing device comprises a data recorder, a processing unit, a transmission unit, and a cloud; the processing unit is connected to the cloud; the cloud is connected to the transmission unit, the data recorder and the transmission unit are both electrically connected to the first temperature probe and the second temperature probe; the transmission unit sends the collected data from the first temperature probe and the second temperature probe to the cloud for storage; the processing unit retrieves and analyzes the stored data, and then stores the processed data again in the cloud; the data recorder displays the temperature data of the first temperature probe and the second temperature probe.

The variable voltage control device comprises a heater and a temperature controller; the heater is connected to the cylindrical heating probe through the temperature controller.

Referring to FIG. 1, a measurement method for thermal characteristics of frozen soil based on the thermal pulse method comprises:
S101. based on the constant temperature heating of the cylindrical heating probe by the heater, the first temperature probe detects the temperature of the cylindrical heating probe, and the second temperature probe detects the temperature of the frozen soil, the data is then uploaded to the cloud through the transmission unit, while the data recorder displays the temperature data of the cylindrical heating probe and the frozen soil in real-time;
S102. when the cylindrical heating probe reaches the target temperature, the temperature controller maintains the temperature of the cylindrical heating probe at a constant value;
when the temperature reading of the cylindrical heating probe drops to the preset lower temperature limit, the heater power supply is turned on, and heating is performed by using short pulses with low energy output power; after the heating period ends, the heater power supply is turned off to keep the temperature of the cylindrical heating probe between the preset upper and lower temperature limits; this step is continuously repeated until the frozen soil sample measurement is completed;
S103. under the heating of the cylindrical heating probe, a temperature breakthrough curve of the frozen soil sample and energy received by the frozen soil sample are obtained, thereby obtaining thermal diffusivity of the frozen soil sample;
the temperature breakthrough curve of the frozen soil sample is obtained as follows:
one-dimensional transient heat conduction process in the cylindrical coordinate system is described as follows:

$$\frac{{\partial }^{2} T}{\partial {r}^{2}} + \frac{1}{r} \frac{\partial T}{\partial r} = \frac{1}{D} \frac{\partial T}{\partial t} ( r > {r}_{e} , t > 0 )$$

wherein T is temperature measured in the soil by the second temperature probe; r is a radial distance from the second temperature probe to the cylindrical heating probe; t is time for measuring the temperature breakthrough curve of the frozen soil; effective radius rₑ is a radial distance between the second temperature probe and the first temperature probe; minimum value of rₑ is equal to the sum of the radii of the first temperature probe and the second temperature probe; and D is volumetric thermal diffusivity;
when heating the frozen soil by using a constant-temperature heat source, target temperature T₁ of the frozen soil is higher than initial temperature T₀ but lower than the freezing point;
while satisfying both initial condition (T(r,0)=T₀(T₀<0)) and boundary condition (T(rₑ,t)=T₁ (T₀<T_{1<0})), the solution in Laplace space is as follows:

$$\overline{T} ( r , s ) = \frac{( {T}_{1} - {T}_{0} ) {K}_{0} ( q r )}{{sK}_{0} ( {qr}_{e} )}$$

wherein K₀ is zero-ordered modified Bessel function of the second kind;

$${q}^{2} = \frac{s}{D}$$

wherein s is time t in the Laplace space after is T(r,t) Laplace transformed;
by performing the numerical inverse Laplace transform, the spatial distribution T(r,t) of temperature in frozen soil at any time can be obtained;

Root Mean Square Error (RMSE) is used to evaluate the fitting of the temperature breakthrough curve, specifically:

$$RMSE = \sqrt{\frac{{{Σ}_{i = 1}^{N} ( {T}_{i} - \hat{{T}_{ι}} )}^{2}}{N}}$$

wherein subscript i is the sequence of measured temperature responses; N is the total number of measured temperatures; T and \hat{T} respectively represent measured temperatures and predicted temperatures.

The energy received by the frozen soil sample, specifically:

$${Q}_{1} ( t ) = {\sum }_{i = 1}^{M} ( {T}_{i} - {T}_{0} ) {ρ}_{b} {c}_{b} π ( {r}_{i + 1}^{2} - {r}_{i}^{2} )$$

wherein subscript i represents the i-th discrete spatial interval after the measurement space is discretized into M spatial intervals in the radial direction, with the spatial range covering the radial distance from r=rₑ to T=T₀;
S104. based on the heating time of the cylindrical heating probe and the heat output during that heating time, the total heat output of the cylindrical heating probe is calculated, specifically:
total output energy Q₂(t) of the cylindrical heating probe is measured by the temperature controller; heating cycle consists of multiple pulsed heating intervals with a given power Pⱼ and heating period tpⱼ, so the total delivered energy is the sum of the energy delivered in each cycle, as shown below:

$${Q}_{2} ( t ) = \frac{1}{L} {\sum }_{j = 1}^{N} {P}_{j} t {p}_{j}$$

wherein L represents length of the cylindrical heating probe.
S105. based on the energy received by the frozen soil sample, the total delivered energy of the cylindrical heating probe, and the volumetric diffusivity of the frozen soil sample, the volumetric heat capacity is obtained, specifically:
from the volumetric heat capacity C_{b}=ρₚc_{b}, the following formula can be obtained:

$${C}_{b} = \frac{{Σ}_{j = 1}^{N} {P}_{j} t {p}_{j}}{L {Σ}_{i = 1}^{M} ( {T}_{i} - {T}_{0} ) π ( {r}_{i + 1}^{2} - {r}_{i}^{2} )}$$

wherein ρ_{b} represents density of medium; c_{b} represents specific heat capacity of the medium;
S106. based on the thermal diffusivity and the volumetric heat capacity, the thermal conductivity is obtained, specifically:
volumetric thermal conductivity of soil:

$${λ}_{b} = D {C}_{b} .$$

When other substances are doped into the frozen soil, and in the case of quartz sand samples being doped, total heat capacity of the quartz sand samples is as follows:

$$C = {n}_{i} {c}_{i} + {n}_{ℊ} {c}_{ℊ} + {n}_{s} {c}_{s}$$

$$ρ = {n}_{i} {ρ}_{i} + {n}_{ℊ} {ρ}_{ℊ} + {n}_{s} {ρ}_{s}$$

wherein n represents volume fraction of components in frozen soil, cᵢ represents specific heat capacity of ice; c_{g} represents specific heat capacity of air; and cₛ represents specific heat capacity of solid; ρᵢ represents volume density of ice; ρ_{g} represents volume density of air; ρₛ represents volume density of solid;
in saturated frozen soil, gaseous term is omitted; based on the total heat capacity C, as well as the specific heat capacities of the solid and ice, the respective volume fractions can be obtained.

Thermal conductivity of λ_{g} dried quartz sand is as follows:

$${λ}_{b 1} = 7 . {5}^{1 - Φ} 0.5 {{1}^{Φ} [ ( 1 - {λ}_{g} ) S r + {λ}_{g} ]}^{0.78 Φ}$$

wherein, Φ represents porosity, Sr represents residual water saturation, 7.5 is thermal conductivity value of quartz grains, 0.51 is thermal conductivity value of water, and λ_{g} is volumetric thermal conductivity of air;
when the pore space of quartz sand is filled with additional components, its content is estimated by the following formula for volumetric thermal conductivity:

$${λ}_{b 2} = {λ}_{qtz}^{{n}_{q t z}} {λ}_{x}^{{n}_{x}}$$

wherein n_{qtz}+nx=1; λ_{qtz}, nₓ and n_{gtz}, λₓ respectively represent the thermal conductivities and volume fractions of quartz sand and the additional components.

### Embodiment

the invention involves a frozen soil sample containing ice, quartz sand, and a mixture; the quartz sand used has a particle size of 50 mesh, composed of Sio₂ more than 99.8%, with a porosity of 0.37. The experiment is conducted with consistent settings as follows: (1) preparing a cylindrical sample with a diameter of 21 cm and a height of 14 cm, and storing it in a refrigerator throughout the experiment to maintain a stable surface and environmental temperature; (2) before the freezing process, installing the cylindrical heating probe, the first temperature probe, and the second temperature probe into the sample to prevent air retention at the probe-sample interface during the drilling process; (3) using a heating probe with a diameter of 6 mm, length of 10 cm, and an output power of 150 W; (4) the temperature of the heating source is manually adjusted by a voltage regulator, as shown in FIG. 1; (5) unless otherwise specified, it is assumed that the physical properties of the sample components remain unchanged regardless of temperature. All samples undergo a pretreatment (freezing/thawing) for more than 24 hours before measurement.

The first temperature probe and the second temperature probe continuously track the temperatures of the cylindrical heating probe and the sample; the vertical position of the tip of the first temperature probe is located near the geometric center of the cylindrical heating probe; a data recorder is used to record and store the temperature measurements from both the first temperature probe and second temperature probe. Temperature readings are taken at 1-second intervals to control the energy delivery of the heater. The temperature-time sequence data collected by the temperature probes is analyzed, assuming that the surface temperature of the heat source remains constant, so as to estimate the thermal properties of the soil. Both the first temperature probe and second temperature probe are calibrated by using ice water and boiling water.

The energy delivery intensity is adjusted by using a voltage regulator to facilitate temperature control of the heat source. Specifically, constant temperature of the heat source is achieved through the following steps:
based on the temperature heating of the cylindrical heating probe by the heater with constant power, the first temperature probe detects the temperature of the cylindrical heating probe, and the second temperature probe detects the temperature of the frozen soil, the data is then uploaded to the cloud through the transmission unit, while the data recorder displays the temperature data of the cylindrical heating probe and the frozen soil in real-time;
when the cylindrical heating probe reaches the target temperature, the heater power supply is turned off, and the energy delivery rate of the heater is reduced through the voltage regulator, thereby maintaining the temperature of the cylindrical heating probe constant;
when the temperature reading of the cylindrical heating probe drops to the preset lower temperature limit, the heater power supply is turned on, and heating is performed by using short pulses with low energy output power; after the heating period ends, the heater power supply is turned off to keep the temperature of the cylindrical heating probe between the preset upper and lower temperature limits; this step is continuously repeated until the frozen soil sample is thawed.
under the heating of the cylindrical heating probe, a temperature breakthrough curve of the frozen soil sample and energy received by the frozen soil sample are obtained, thereby obtaining thermal diffusivity of the frozen soil sample;
based on heating time of the cylindrical heating probe and the heat output during that heating time, total heat output of the cylindrical heating probe is calculated;
based on the energy received by the frozen soil sample, total delivered energy of the cylindrical heating probe, and volumetric diffusivity of the frozen soil sample, volumetric heat capacity is obtained; based on the thermal diffusivity and volumetric heat capacity, thermal conductivity is obtained.

In this invention, the estimation of thermal properties of frozen soil depends on the description of the heat transfer process, which is primarily controlled by thermal conduction within a mixture of stationary solid phase, water phase, and gas phase. The model used to describe the propagation of conductive heat assumes that (1) thermal conduction is the sole mechanism driving heat transfer in frozen soil; and (2) the physical properties of frozen soil are isotropic and homogeneous, meaning there is no variation in spatial and temporal properties. Therefore, when significant changes in soil thermal properties occur during freezing and thawing processes, this model fails to estimate transient temperature variations.

One-dimensional transient heat conduction process in the cylindrical coordinate system is described as follows:

$$\frac{{\partial }^{2} T}{\partial {r}^{2}} + \frac{1}{r} \frac{\partial T}{\partial r} = \frac{1}{D} \frac{\partial T}{\partial t} ( r > {r}_{e} , t > 0 ) \qquad ( 1 )$$

wherein T is temperature measured in the soil by the second temperature probe; r is a radial distance from the second temperature probe to the cylindrical heating probe; t is time for measuring the temperature breakthrough curve of the frozen soil; effective radius rₑ is a radial distance between the second temperature probe and the first temperature probe; minimum value of rₑ is equal to the sum of the radii of the first temperature probe and the second temperature probe; and D is volumetric thermal diffusivity;
when heating the frozen soil by using a constant-temperature heat source, target temperature T₁ of the frozen soil is higher than initial temperature T₀ but lower than the freezing point;
while satisfying both initial condition (T(r,0)=T₀(T₀<0)) and boundary condition (T(rₑ,t)=T₁ (T₀<T₁<0)), the solution in Laplace space is as follows:

$$\overline{T} ( r , s ) = \frac{( {T}_{1} - {T}_{0} ) {K}_{0} ( q r )}{s {K}_{0} ( q {r}_{e} )} \qquad ( 2 )$$

wherein K₀ is zero-ordered modified Bessel function of the second kind;

$${q}^{2} = \frac{s}{D} \qquad ( 3 )$$

wherein s is time t in the Laplace space after is T(r,t) Laplace transformed;
Root Mean Square Error (RMSE) is used to evaluate the fitting of the temperature breakthrough curve, specifically:

$$RMSE = \sqrt{\frac{{{Σ}_{i = 1}^{N} ( {T}_{i} - {\hat{T}}_{ι} )}^{2}}{N}} \qquad ( 4 )$$

wherein subscript i is the sequence of measured temperature responses; N is the total number of measured temperatures; T and \hat{T} respectively represent measured temperatures and predicted temperatures.

Through given specific values of r, rₑ, and t, the temperature breakthrough curve is fitted to field or experimental temperatures to obtain the volumetric diffusivity

$$D = \frac{{λ}_{b}}{{ρ}_{b} {c}_{b}} ;$$

it is straightforward to plot the spatial temperature range as a function of radial distance.

The energy received by the frozen soil sample, specifically:

$${Q}_{1} ( t ) = {\sum }_{i = 1}^{M} ( {T}_{i} - {T}_{0} ) {ρ}_{b} {c}_{b} π ( {r}_{i + 1}^{2} - {r}_{i}^{2} ) \qquad ( 5 )$$

wherein subscript i represents the i-th discrete spatial interval after the measurement space is discretized into M spatial intervals in the radial direction, with the spatial range covering the radial distance from r=rₑ to T=T₀;
based on the heating time of the cylindrical heating probe and the heat output during that heating time, the total heat output of the cylindrical heating probe is calculated, specifically:

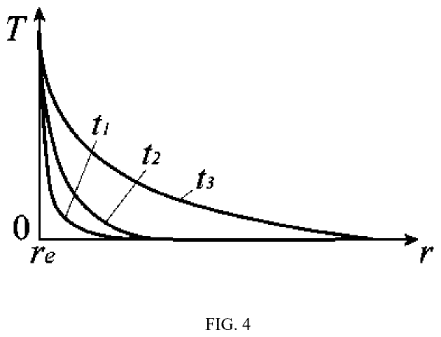

Referring FIG. 3 and FIG. 4, heating cycle consists of multiple pulsed heating intervals with a given power Pⱼ and heating period tpⱼ, so the total delivered energy is the sum of the energy delivered in each cycle, as shown below:

$${Q}_{2} ( t ) = \frac{1}{L} {\sum }_{j = 1}^{N} {P}_{j} {tp}_{j} \qquad ( 6 )$$

wherein L represents length of the cylindrical heating probe. ILS model is the frozen soil measurement device shown in FIG. 1.
based on the energy received by the frozen soil sample, the total delivered energy of the cylindrical heating probe, and the volumetric diffusivity of the frozen soil sample, the volumetric heat capacity is obtained, specifically:
from the volumetric heat capacity C_{b}=ρ_{b}c_{b}, the following formula can be obtained:

$${C}_{b} = \frac{{\sum }_{j = 1}^{N} {P}_{j} {tp}_{j}}{L {\sum }_{i = 1}^{M} ( {T}_{i} - {T}_{0} ) π ( {r}_{i + 1}^{2} - {r}_{i}^{2} )} \qquad ( 7 )$$

wherein ρ_{b} represents density of medium; c_{b} represents specific heat capacity of the medium;
volumetric thermal conductivity of soil:

$${λ}_{b} = {DC}_{b} \qquad ( 8 )$$

when other substances are doped into the frozen soil, and in the case of quartz sand samples being doped, thermal characteristics of the quartz sand samples is as follows:

$$ρ = {n}_{i} {ρ}_{i} + {n}_{g} {ρ}_{g} + {n}_{s} {ρ}_{s} \qquad ( 9 )$$

$$C = {n}_{i} {c}_{i} + {n}_{g} {c}_{g} + {n}_{s} {c}_{s} \qquad ( 10 )$$

wherein n represents volume fraction of components in frozen soil, pᵢ represents volume density of ice; ρ_{g} represents volume density of air; ρₛ represents volume density of solid; cᵢ represents specific heat capacity of ice; c_{g} represents specific heat capacity of air; and cₛ represents specific heat capacity of solid;
thermal conductivity of dried quartz sand is as follows:

$${λ}_{b 1} = 7 . {5}^{1 - Φ} 0.5 {{1}^{Φ} [ ( 1 - {λ}_{g} ) Sr + {λ}_{g} ]}^{0.78 Φ} \qquad ( 11 )$$

wherein, Φ represents porosity, Sr represents residual water saturation, 7.5 is thermal conductivity value of quartz grains, 0.51 is thermal conductivity value of water, and λ_{g} is volumetric thermal conductivity of air;
when the pore space of quartz sand is filled with additional components, its content is estimated by the following formula for volumetric thermal conductivity:

$${λ}_{b 2} = {λ}_{qtz}^{{n}_{qtz}} {λ}_{x}^{{n}_{x}} \qquad ( 12 )$$

wherein n_{qtz}+nx=1; λ_{qtz}, nₓ and n_{qtz}, λₓ respectively represent the thermal conductivities and volume fractions of quartz sand and the additional components.

This invention effectively minimizes the melting process near the freezing point (−5 to 0° C.) by maintaining the maximum domain temperature, i.e., the temperature of the cylindrical heating probe, below the freezing point. In colder frozen soil (temperatures below −5° C.), the melting process can be avoided by setting a maximum temperature well below the freezing point. Therefore, the sensitivity of the input variables in the proposed model was analyzed by using the benchmark case parameters, as shown in Table 1.

TABLE 1
Benchmark Case Parameters for Sensitivity Analysis

| Parameter | Symbol | Value | Unit |
|---|---|---|---|
| Volumetric Thermal | λ | 1.5 | W/(m · K) |
| Conductivity | | | |
| Bulk Density | ρ | 1,000.0 | kg/m³ |
| Volumetric Specific | c | 1,500.0 | J /(kg · ° C.) |
| Heat Capacity | | | |
| Temperature Change | T | 10 | k |
| Effective Radius of | r | 0.005 | m |
| Heat Source | | | |
| Length of Heat | L | 0.1 | m |
| Source | | | |
| Simulation Time | t | 500 | s |

The above embodiments are only used to illustrate the technical solutions of the invention rather than to limit the invention. Although the invention has been described in detail with reference to the above embodiments, ordinary technicians in the relevant field should understand that the specific embodiments of the invention can still be modified or replaced by equivalents, and any modification or equivalent replacement that does not deviate from the spirit and scope of the invention should be included in the protection scope of the claims of the invention.

1. A measurement device for thermal characteristics of frozen soil based on a thermal pulse method, comprising a testing assembly, a variable voltage control device, and a data processing device;
the testing assembly comprises a cylindrical heating probe, a first temperature probe, and a second temperature probe; the cylindrical heating probe, the first temperature probe, and the second temperature probe are all placed in the frozen soil sample to be measured; the variable voltage control device is connected to the cylindrical heating probe and the first temperature probe, providing energy to the cylindrical heating probe, and the first temperature probe records the surface temperature of the heating probe; the data processing device is connected to the second temperature probe; the second temperature probe measures the temperature changes of the frozen soil.

2. The measurement device for thermal characteristics of frozen soil based on the thermal pulse method of claim 1, wherein the first temperature probe, the second temperature probe, and the cylindrical heating probe are arranged parallel to each other; the first temperature probe, the second temperature probe, and the cylindrical heating probe are arranged in the frozen soil sample to be measured in either a horizontal or vertical manner.

3. The measurement device for thermal characteristics of frozen soil based on the thermal pulse method of claim 2, wherein the data processing device comprises a data recorder, a processing unit, a transmission unit, and a cloud; the processing unit is connected to the cloud; the cloud is connected to the transmission unit, the data recorder and the transmission unit are both electrically connected to the first temperature probe and the second temperature probe; the transmission unit sends the collected data from the first temperature probe and the second temperature probe to the cloud for storage; the processing unit retrieves and analyzes the stored data, and then stores the processed data again in the cloud; the data recorder displays the temperature data of the first temperature probe and the second temperature probe.

4. The measurement device for thermal characteristics of frozen soil based on the thermal pulse method of claim 3, wherein the variable voltage control device comprises a heater and a temperature controller; the heater is connected to the cylindrical heating probe through the temperature controller.

5. A measurement method for thermal characteristics of frozen soil based on the thermal pulse method, using the measurement device for thermal characteristics of frozen soil based on the thermal pulse method as claimed in claim 4, wherein the measurement method comprises:
based on the constant temperature heating of the cylindrical heating probe by the heater, the first temperature probe detects the temperature of the cylindrical heating probe, and the second temperature probe detects the temperature of the frozen soil, the data is then uploaded to the cloud through the transmission unit, while the data recorder displays the temperature data of the cylindrical heating probe and the frozen soil in real-time;
when the cylindrical heating probe reaches the target temperature, the temperature controller maintains the temperature of the cylindrical heating probe at a constant value;
under the heating of the cylindrical heating probe, a temperature breakthrough curve of the frozen soil sample and energy received by the frozen soil sample are obtained, thereby obtaining thermal diffusivity of the frozen soil sample;
based on heating time of the cylindrical heating probe and the heat output during that heating time, total heat output of the cylindrical heating probe is calculated;
based on the energy received by the frozen soil sample, total delivered energy of the cylindrical heating probe, and volumetric diffusivity of the frozen soil sample, volumetric heat capacity is obtained;
finally, based on the thermal diffusivity and volumetric heat capacity, thermal conductivity is obtained.

6. The measurement method for thermal characteristics of frozen soil based on the thermal pulse method of claim 5, wherein when the cylindrical heating probe controls the energy output power of the heater through the temperature controller to keep the temperature of the cylindrical heating probe constant, specifically: when the temperature reading of the cylindrical heating probe drops to the preset lower temperature limit, the heater power supply is turned on, and heating is performed by using short pulses with low energy output power; after the heating period ends, the heater power supply is turned off to keep the temperature of the cylindrical heating probe between the preset upper and lower temperature limits; this step is continuously repeated until the frozen soil sample measurement is completed.

7. The measurement method for thermal characteristics of frozen soil based on the thermal pulse method of claim 6, wherein the temperature breakthrough curve of the frozen soil sample is obtained as follows:
one-dimensional transient heat conduction process in the cylindrical coordinate system is described as follows:
$$\frac{{\partial }^{2} T}{\partial {r}^{2}} + \frac{1}{r} \frac{\partial T}{\partial r} = \frac{1}{D} \frac{\partial T}{\partial t} ( r > {r}_{e} , t > 0 )$$
wherein T is temperature measured in the soil by the second temperature probe; r is a radial distance from the second temperature probe to the cylindrical heating probe; t is time for measuring the temperature breakthrough curve of the frozen soil; effective radius rₑ is a radial distance between the second temperature probe and the first temperature probe; minimum value of rₑ is equal to the sum of the radii of the first temperature probe and the second temperature probe; and D is volumetric thermal diffusivity;
when heating the frozen soil by using a constant-temperature heat source, target temperature T₁ of the frozen soil is higher than initial temperature T₀ but lower than the freezing point;
while satisfying both initial condition (T(r,0)=T₀(T₀<0)) and boundary condition (T(rₑ,t)=T₁(T₀<T₁<0)), the solution in Laplace space is as follows:
$$\overline{T} ( r , s ) = \frac{( {T}_{1} - {T}_{0} ) {K}_{0} ( qr )}{{sK}_{0} ( {qr}_{e} )}$$
wherein K₀ is zero-ordered modified Bessel function of the second kind;
$${q}^{2} = \frac{s}{D}$$
wherein s is time t in the Laplace space after is T(r,t) Laplace transformed;
by performing the numerical inverse Laplace transform, the spatial distribution T(r,t) of temperature in frozen soil at any time can be obtained;
Root Mean Square Error (RMSE) is used to evaluate the fitting of the temperature breakthrough curve, specifically:
$$RMSE = \sqrt{\frac{{\sum }_{i = 1}^{N} {( {T}_{i} - {\hat{T}}_{ι} )}^{2}}{N}}$$
wherein subscript i is the sequence of measured temperature responses; N is the total number of measured temperatures; T and \hat{T} respectively represent measured temperatures and predicted temperatures.

8. The measurement method for thermal characteristics of frozen soil based on the thermal pulse method of claim 7, wherein the energy received by the frozen soil sample, specifically:
$${Q}_{1} ( t ) = {\sum }_{i = 1}^{M} ( {T}_{i} - {T}_{0} ) {ρ}_{b} {c}_{b} π ( {r}_{i + 1}^{2} - {r}_{i}^{2} )$$
wherein subscript i represents the i-th discrete spatial interval after the measurement space is discretized into M spatial intervals in the radial direction, with the spatial range covering the radial distance from r=rₑ to T=T₀;
based on the heating time of the cylindrical heating probe and the heat output during that heating time, the total heat output of the cylindrical heating probe is calculated, specifically:
total output energy Q₂(t) of the cylindrical heating probe is measured by the temperature controller; heating cycle consists of multiple pulsed heating intervals with a given power Pⱼ and heating period tₚ_{ⱼ}, so the total delivered energy is the sum of the energy delivered in each cycle, as shown below:
$${Q}_{2} ( t ) = \frac{1}{L} {\sum }_{j = 1}^{N} {P}_{j} {tp}_{j}$$
wherein L represents length of the cylindrical heating probe.

9. The measurement method for thermal characteristics of frozen soil based on the thermal pulse method of claim 8, wherein based on the energy received by the frozen soil sample, the total delivered energy of the cylindrical heating probe, and the volumetric diffusivity of the frozen soil sample, the volumetric heat capacity is obtained, specifically:
from the volumetric heat capacity C_{b}=ρₚc_{b}, the following formula can be obtained:
$${C}_{b} = \frac{{\sum }_{j = 1}^{N} {P}_{j} {tp}_{j}}{L {\sum }_{i = 1}^{M} ( {T}_{i} - {T}_{0} ) π ( {r}_{i + 1}^{2} - {r}_{i}^{2} )}$$
wherein ρ_{b} represents density of medium; c_{b} represents specific heat capacity of the medium;
based on the thermal diffusivity and the volumetric heat capacity, the thermal conductivity is obtained, specifically:
volumetric thermal conductivity of soil:
$${λ}_{b} = {DC}_{b} .$$

10. The measurement method for thermal characteristics of frozen soil based on the thermal pulse method of claim 9, wherein when other substances are doped into the frozen soil, and in the case of quartz sand samples being doped, total heat capacity of the quartz sand samples is as follows:
$$C = {n}_{i} {c}_{i} + {n}_{g} {c}_{g} + {n}_{s} {c}_{s}$$
wherein n represents volume fraction of components in frozen soil, cᵢ represents specific heat capacity of ice; c_{g} represents specific heat capacity of air; and cₛ represents specific heat capacity of solid;
in saturated frozen soil, gaseous term is omitted; based on the total heat capacity C, as well as the specific heat capacities of the solid and ice, the respective volume fractions can be obtained.
